(19) United States
(12) Patent Application Publication
(10) Pub. No.: US 2007/0275422 A1
(43) Pub. Date: Nov. 29, 2007

(54) Methods and Compositions for Detecting Pancreatic Disease

(75) Inventor: Anson Lowe, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: THE BOARD OF TRUSTEES OF THE LELAND STANFORD JR. U, Palo Alto, CA (US)

(21) Appl. No.: 11/632,260

(22) PCT Filed: Jul. 26, 2004
(86) PCT No.: PCT/US04/24108

§ 371 Date: Jul. 23, 2007

(51) Int. Cl. G01N 33/574

(52) U.S. Cl. 435/7.23; 436/501

(57) ABSTRACT

Methods and compositions for at least determining whether a human subject has a pancreatic disease are provided. In practicing the subject methods, a sample from a subject is assayed for a pancreatic glycoprotein (GP2) analyte to determine whether the subject at least has the pancreatic disease. Also provided are kits, systems, and devices for practicing the subject methods.

### BACKGROUND OF THE INVENTION

The pancreas is a large, elongated gland situated transversely behind the stomach, between the spleen and the duodenum. Common disorders of the pancreas include pancreatitis, which includes acute and chronic pancreatitis, and pancreatic cancer.

Pancreatitis is an inflammation of the pancreas accompanied by autodigestion of pancreatic tissue by its own enzymes. Pancreatitis may be either acute or chronic. Acute pancreatitis is associated with a sudden onset of abdominal pain, nausea, and vomiting. Predisposing conditions for acute pancreatitis include chronic alcoholism, gallstones, hypercalcemia, hyperlipoproteinemia, blunt abdominal trauma, and penetrating peptic ulcer. Predisposition may also be inherited as an autosomal dominant trait.

Chronic pancreatitis, which may be accompanied by recurrent attacks of acute pancreatitis, is usually associated with recurrent, chronic abdominal pain, progressive fibrosis and loss of exocrine function (e.g., malabsorption) and endocrine (diabetes mellitus) function, though the disease may also be asymptomatic. Although the specific pathogenesis of chronic pancreatitis is unknown, most cases of the disease suggest that obstruction of the pancreatic ducts may play a crucial role. Forms of acute or chronic pancreatitis include acute hemorrhagic pancreatitis, chronic calcific (calcareous) pancreatitis, centrilobular pancreatitis, and perilobular pancreatitis.

Pancreatitis is conventionally diagnosed by physical examination and by evaluation of levels of amylase, lipase, or trypsin in serum, or the ratio of amylase to creatinine in serum. None of these conventional diagnostic tests is specific for pancreatitis. For example, increased serum amylase may also be due to cholecystitis, hepatitis, intestinal obstruction, mesenteric thrombosis, parotitis, perforated duodenal ulcer, or a ruptured aortic aneurysm. Increased levels of serum trypsin can be caused by chronic renal failure. Similarly, the ratio of amylase to creatinine in serum may be elevated by uremia or acute tubular damage.

The initial symptoms of pancreatic cancer are usually nonspecific (e.g., abdominal pain and weight loss) and are frequently disregarded. The deep anatomic location of the pancreas makes detection of small localized tumors unlikely during the routine abdominal examination. Even in patients with confirmed pancreatic cancer, an abdominal mass is palpable in only 15-25% of cases. Diagnosis of pancreatic cancer is further complicated by the occurrence of dysplastic cells, i.e., abnormal cells that are not cancerous. Thus, even a biopsy can result in an erroneous diagnosis. Biopsy diagnoses may also be complicated by other underlying pancreatic disorders such as diabetes or pancreatitis. Unfortunately, because pancreatic cancer is generally very aggressive, some 80-90% of patients have regional and distant metastases by the time they are diagnosed and as such, pancreatic cancer is associated with a high mortality rate.

Accordingly, there is a need for the development of a specific and sensitive assay for determining whether a human subject at least has a pancreatic disease, such as pancreatitis or pancreatic cancer. The present invention addresses this need.

Relevant Literature

U.S. patents of interest include: U.S. Pat. No. 5,663,315. Also of interest is: Lowe et al., 1994, Gastroenterology 107(6):1819-1827; Fukuoka et al., 1991, PNAS 88:2898-2902; Fukuoka et al., 1992, PNAS 89:1189-1193; Fritz et al., 2002, Pancreas 24(4):336-343; Wong et al., 1996, Gene 171 (2) 311-312; Hoops et al., 1991, J. Biol. Chem. 266:4257-4263; and MacDonald et al., 1972, Biochem. Biophys. Res. Comm. 49:377-382; Leblond et al., 1989, J. Immunology, 124(1):71-75.

### SUMMARY OF THE INVENTION

Methods and compositions for determining whether a human subject at least has a pancreatic disease are provided. In practicing the subject methods, a sample from a subject is assayed for a pancreatic glycoprotein (GP2) analyte to determine whether the subject at least has the pancreatic disease. Also provided are kits, systems, and devices for practicing the subject methods.

These and other objects, advantages, and features of the invention will become apparent to those persons skilled in the art upon reading the details of the invention as more fully described below.

### Features of the Invention

One feature of the invention provides a method of determining whether a human subject at least has a pancreatic disease by assaying a sample from the human subject for a pancreatic glycoprotein (GP2) analyte to determine whether the human subject at least has the pancreatic disease. In such methods the pancreatic disease may be acute pancreatitis, chronic pancreatitis, or pancreatic cancer. In such methods, the sample may be a blood sample, e.g., whole blood or a fraction thereof, e.g., serum, or other physiological sample, e.g., urine. In some embodiments, the GP2 analyte is human GP2 long isoform.

In some embodiments, the assaying includes determining the amount of the GP2 analyte in the sample. In such embodiments, the amount may be determined by comparing a detected signal or amount to a reference.

In other embodiments, the assaying employs a GP2 affinity reagent. In such embodiments, the affinity reagent may include a GP2 specific antibody or binding fragment thereof. In such embodiments, the affinity reagent may be immobilized on a surface of a solid support. In such embodiments, the affinity reagent may include a detectable label. In such embodiments, the assaying may employ at least two different GP2 affinity reagents.

A feature of certain embodiments is that the assay is a pancreatitis assay, e.g., either acute or chronic, where in certain such embodiments, the assay is more accurate than an amylase assay.

In yet another embodiments, the method is a method of determining severity of the pancreatic disease of said subject. In yet another embodiments, the method is a method of monitoring progression of the pancreatic disease of said subject.

Another feature of the invention provides a method of treating a human subject for a pancreatic disease by determining at least whether the human subject suffers from the pancreatic disease by assaying a sample from the human subject for a pancreatic glycoprotein (GP2); and identifying a treatment protocol for the human subject based on results from the determining step.

Yet another feature of the invention provides a kit for use in determining at least whether a subject suffers from a pancreatic disease, including reagents for assaying a sample for a GP2 analyte; and a reference. Such kits may further include a sample obtainment element. In some embodiments, the GP2 analyte is GP2 protein long isoform. In some embodiments, the reagents include at least one GP2 affinity reagent. In such embodiments, the at least one GP2 affinity reagent is immobilized on a surface of a solid support.

Yet another feature of the invention provides a system for use in determining at least whether a subject suffers from a pancreatic disease, including reagents for assaying a sample for a GP2 analyte; and a reference. Such systems may further include a sample obtainment element. In some embodiments, the GP2 analyte is GP2 protein long isoform. In other embodiments, the reagents include at least one GP2 affinity reagent. In yet other embodiments, the least one GP2 affinity reagent is immobilized on a surface of a solid support.

Yet another feature of the invention provides a device for use in determining at least whether a subject suffers from a pancreatic disease, including a GP2 affinity reagent immobilized on a surface of a solid support. In some embodiments, the GP2 affinity reagent includes an antibody or binding fragment thereof.

### BRIEF DESCRIPTION OF THE DRAWINGS

The invention is best understood from the following detailed description when read in conjunction with the accompanying drawings. It is emphasized that, according to common practice, the various features of the drawings may not be to-scale. On the contrary, the dimensions of the various features may be arbitrarily expanded or reduced for clarity. Included in the drawings are the following figures:

FIG. 1 is an immunoblot of human GP2 probed with mouse monoclonal antibody 2G2/2E10 (mAb) and a rabbit anti-human GP2 polyclonal antisera (pAb). The expected 97 KDa band is detected. The blots consist of tissue culture media containing recombinant human GP2 produced by Chinese Hamster Ovary cells.

FIG. 2 is a graph showing the distribution of GP2 and amylase levels according to pancreatic disease. Medians of GP2 and amylase levels in each group are indicated with a bar. Amylase levels were not determined for patients with pancreatic cancer since it is well known that amylase levels have no diagnostic or prognostic utility with respect to pancreatic cancer.

FIG. 3 shows Receiver Operator Characteristic (ROC) curves for the GP2 (solid lines) and amylase (dashed lines) assays in acute pancreatitis (top panel), chronic pancreatitis (middle panel), and pancreatitic cancer (bottom panel). The control used was the combined normal and non-pancreatic disease groups. Serum amylase levels were not available for the majority of patients with pancreatic cancer.

FIG. 4 shows the cumulative rate of elevated GP2 (solid line) and amylase (dash line) levels in patients with acute pancreatitis. Blood samples were collected daily from 13 patients with acute pancreatitis from admission to discharge and GP2 and amylase levels were determined. Cutoff points used for GP2 and amylase were 4.5 pmol/L and 134 IU/L respectively. The cumulative positive rates were calculated using Kaplan-Meier survival analysis. The difference between the two curves was significant (p<0.005 by Mantel-Cox test).

### DEFINITIONS

By “pancreatitis” is meant acute or chronic inflammation of the pancreas accompanied by autodigestion of pancreatic tissue by its own enzymes. Pancreatitis may be symptomatic or asymptomatic.

By “acute pancreatitis” is meant pancreatitis characterized by sudden onset of abdominal pain, nausea, and vomiting. Acute pancreatitis may occur in the setting of chronic pancreatitis.

By “chronic pancreatitis” is meant pancreatitis normally characterized by chronic recurrent abdominal pain, progressive fibrosis, and loss of exocrine and endocrine function. Chronic pancreatitis may be associated with episodes of acute pancreatitis.

The term “pancreatic cancer” encompasses benign or malignant forms of pancreatic cancer, as well as any particular type of cancer arising from cells of the pancreas (e.g., duct cell carcinoma, acinar cell carcinoma, papillary carcinoma, adenosquamous carcinoma, undifferentiated carcinoma, mucinous carcinoma, giant cell carcinoma, mixed type pancreatic cancer, small cell carcinoma, cystadenocarcinoma, unclassified pancreatic cancers, pancreatoblastoma, and papillary-cystic neoplasm, and the like.).

By “bodily fluid” is meant a naturally occurring fluid of the human body such as serum, plasma, blood, urine, mucus, gastric juices, pancreatic juices, or lymph, particularly blood or blood products and urine.

By “blood sample” is meant a volume of whole blood or fraction thereof, e.g., serum, plasma, etc.

By “disease severity” is meant relative stage of disease progression. Disease severity may be correlated with the impact the disease may have on the patient's overall health or the risk of patient death as a result of disease. The severity of the disease may affect decisions relating to patient treatment subsequent to diagnosis.

By “human pancreatic glycoprotein,” or “human GP2” is meant a Glycoprotein-2 of the human pancreas normally primarily located on the inner leaflet of the zymogen granule (ZG) membranes and the apical plasma membrane of pancreatic acinar cells. Human Glycoprotein-2 exists in two isoforms, the short isoform (GenBank Accession No. D38225) (Fukuoka, et al., 2000, Biochim. Biophys. Acta 1491:376-380) and the long isoform (GenBank Accession No. U36221) (Wong et al., 1996, Gene 171 (2) 311-312).

By “antigenic fragment” of GP2 is meant a portion of GP2 which is capable of binding an antibody generated by immunization of a mammal with GP2 or a fragment thereof. Preferably, the antibodies which specifically bind an epitope of the isolated antigenic fragment will also bind the same epitope in the context of the native protein from which the fragment was derived.

An “affinity reagent” of the subject invention has an analyte binding domain, moiety or component that has a high binding affinity for a target analyte. By high binding affinity is meant a binding affinity of at least about 10⁻⁴ M, usually at least about 10⁻⁶ M or higher, e.g., 10⁻⁹M or higher. The affinity reagent may be any of a variety of different types of molecules, so long as it exhibits the requisite binding affinity for the target protein when present as tagged affinity ligand.

As such, the affinity reagent may be a small molecule or large molecule ligand. By small molecule ligand is meant a ligand ranging in size from about 50 to about 10,000 daltons, usually from about 50 to about 5,000 daltons and more usually from about 100 to about 1000 daltons. By large molecule is meant a ligand ranging in size from about 10,000 daltons or greater in molecular weight.

The small molecule may be any molecule, as well as binding portion or fragment thereof, that is capable of binding with the requisite affinity to the target protein. Generally, the small molecule is a small organic molecule that is capable of binding to the target analyte of interest. The small molecule will include one or more functional groups necessary for structural interaction with the target analyte, e.g., groups necessary for hydrophobic, hydrophilic, electrostatic or even covalent interactions. Where the target analyte is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. The small molecule may also comprise a region that may be modified and/or participate in covalent linkage to a label component, a substrate surface, or other entity, depending on the particular assay protocol being employed, without substantially adversely affecting the small molecule's ability to bind to its target analyte.

Small molecule affinity ligands often comprise cyclical carbon or heterocyclic structures and/or aromatic or polyaromatic structures substituted with one or more of the above functional groups. Also of interest as small molecules are structures found among biomolecules, including peptides, saccharides, fatty acids, steroids, purines, pyrimidines, derivatives, structural analogs or combinations thereof. Such compounds may be screened to identify those of interest, where a variety of different screening protocols are known in the art.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e. a compound diversity combinatorial library. When obtained from such libraries, the small molecule employed will have demonstrated some desirable affinity for the protein target in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

As pointed out, the affinity ligand can also be a large molecule. Of particular interest as large molecule affinity ligands are antibodies, as well as binding fragments and mimetics thereof. Where antibodies are the affinity ligand, they may be derived from polyclonal compositions, such that a heterogeneous population of antibodies differing by specificity are each tagged with the same tag nucleic acid, or monoclonal compositions, in which a homogeneous population of identical antibodies that have the same specificity for the target protein are each tagged with the same tag nucleic acid. As such, the affinity ligand may be either a monoclonal and polyclonal antibody. In yet other embodiments, the affinity ligand is an antibody binding fragment or mimetic, where these fragments and mimetics have the requisite binding affinity for the target protein. For example, antibody fragments, such as Fv, F(ab)₂, Fab′ and Fab may be prepared by cleavage of the intact protein, e.g. by protease or chemical cleavage. Also of interest are recombinantly produced antibody fragments, such as single chain antibodies or scFvs, where such recombinantly produced antibody fragments retain the binding characteristics of the above antibodies. Such recombinantly produced antibody fragments generally include at least the VH and VL domains of the subject antibodies, so as to retain the binding characteristics of the subject antibodies. These recombinantly produced antibody fragments or mimetics of the subject invention may be readily prepared using any convenient methodology, such as the methodology disclosed in U.S. Pat. Nos. 5,851,829 and 5,965,371; the disclosures of which are herein incorporated by reference.

The above described antibodies, fragments and mimetics thereof may be obtained from commercial sources and/or prepared using any convenient technology, where methods of producing polyclonal antibodies, monoclonal antibodies, fragments and mimetics thereof, including recombinant derivatives thereof, are known to those of the skill in the art.

Also suitable for use as binding domains are polynucleic acid aptimers. Polynucleic acid aptamers may be RNA oligonucleotides which may act to selectively bind proteins, much in the same manner as a receptor or antibody (Conrad et al., Methods Enzymol. (1996), 267 (Combinatorial Chemistry), 336-367).

By “binds specifically” is meant high avidity and/or high affinity binding of an antibody to a specific antigen. Antibody binding to its epitope on this specific antigen is stronger than binding of the same antibody to any other epitope, particularly those which may be present in molecules in association with, or in the same sample, as the specific antigen of interest. Antibodies which bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g., by use of appropriate controls.

By ““detectably labeled affinity reagent”, “detectably labeled antibody”, “detectably labeled GP2” or “detectably labeled GP2 fragment” is meant an affinity reagent, e.g., antibody (or antibody fragment which retains binding specificity), GP2, or GP2 polypeptide fragment having an attached detectable label. The detectable label may be attached by chemical conjugation, but where the label is a polypeptide, it could alternatively be attached by genetic engineering techniques. Methods for production of detectably labeled proteins are well known in the art. Detectable labels may be selected from a variety of such labels known in the art, but normally are radioisotopes, fluorophores, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labelling antibodies, and methods for using labeled antibodies to detect an antigen (such as GP2 or GP2 fragments) are well known in the art (see, for example, Harlow and Lane, eds. (Antibodies: A Laboratory Manual (1988) Cold Spring Harbor Laboratory Press, Cold Spring Harbor, N.Y.)).

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION OF THE INVENTION

Methods and compositions for determining whether a human subject at least has a pancreatic disease are provided. In practicing the subject methods, a sample from a subject is assayed for a pancreatic glycoprotein (GP2) analyte to determine whether the subject at least has the pancreatic disease. Also provided are kits, systems, and devices for practicing the subject methods.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an analyte” includes a plurality of such analyte and reference to “the disease” includes reference to one or more disease and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The practice of the present invention will employ, unless otherwise indicated, conventional methods of chemistry, biochemistry, recombinant DNA techniques and virology, within the skill of the art. Such techniques are explained fully in the literature. See, e.g., Fundamental Virology, 2nd Edition, vol. I & II (B. N. Fields and D. M. Knipe, eds.); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.); Oligonucleotide Synthesis (N. Gait, ed., 1984); A Practical Guide to Molecular Cloning (1984).

In further describing the invention, the subject methods are described first in greater detail, followed by a review of representative applications in which the subject methods find use, as well a review of kits, systems and devices that find use in practicing the subject methods.

Methods

As summarized above, the subject invention provides a method of determining whether a human subject at least has a pancreatic disease. The phrase “at least has a pancreatic disease” includes determining whether a human subject has a pancreatic disease (i.e., making an initial diagnosis for the disease) as well determining the severity of the disease, monitoring the progression of the disease or a course of treatment therefore, etc. In one aspect, the subject invention provides methods for determining whether a human subject is suffering from a pancreatic disease. In another aspect, the subject invention provides methods for determining the severity of the pancreatic disease. In yet another aspect, the subject invention provides methods for monitoring progression of the pancreatic disease. By “pancreatic disease” is meant a disease afflicting the human pancreas. Such a disease may include pancreatitis, such as chronic pancreatitis and acute pancreatitis. Such a disease may also include pancreatic cancer. The term “pancreatic cancer” encompasses benign or malignant forms of pancreatic cancer, as well as any particular type of cancer arising from cells of the pancreas.

In determining whether a human subject at least has a pancreatic disease, a sample from the human subject is assayed to determine the level of a pancreatic glycoprotein analyte present in the sample. As used herein, “pancreatic glycoprotein analyte” is meant to include both the long and short isoforms of the pancreatic glycoprotein-2 (GP-2) of the human pancreas and low molecular weight fragments of either the short or the long isoforms of GP-2. A pancreatic glycoprotein may be detected as a monomer, a complex of pancreatic glycoprotein analytes, or as a complex of pancreatic glycoprotein analytes and proteoglycan. The short isoform of GP-2 is a 75 kDa glycoprotein. The nucleic acid sequence for the short isoform of GP-2 is provided in GenBank Accession No. D38225, and further described in Fukuoka et al., Biochim. Biophys. Acta (2000), 1491: 376-380. The long isoform of GP-2 is a 97 kDa glycoprotein. The nucleic acid sequence for the long isoform of GP-2 is provided in GenBank Accession No. U36221, and further described in Wong et al., 1996. Of particular interest in certain embodiments is assaying to determine the level of the long isoform of GP-2 present in a sample from a human subject. As such, in certain embodiments the GP2 analyte of interest for which a given sample is assayed is the human long isoform, and not the short isoform.

As summarized above, in practicing the subject methods a sample from a subject is assayed for a GP2 analyte. The sample that is assayed is a sample that is, or is derived from, any initial source that contains a GP2 analyte. Accordingly, a suitable sample source will be derived from fluids into which the GP2 analyte has been released. Sample sources of interest include, but are not limited to, many different bodily fluids, e.g., serum, plasma, blood, urine, mucus, gastric juices, pancreatic juices, and lymph, particularly blood or blood products and urine. Sample sources of particular interest include blood samples, e.g., whole blood, serum or plasma, and urine. A sample volume of blood, serum, or urine between about 2 μl to about 2,000 μl is sufficient for determining the level of a GP2 analyte. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μl.

Appropriate control samples for the assay include blood, serum, or urine collected from human subjects who do not have a pancreatic disease (i.e., a negative control), or samples which contain a known, predetermined amount of a GP2 analyte (i.e., a positive control).

In many embodiments, a suitable initial source for the human sample is a blood sample. As such, the sample employed in the subject assays is generally a blood-derived sample. The blood derived sample may be derived form whole blood or a fraction thereof, e.g., serum, plasma, etc., where in some embodiments the sample is derived from blood allowed to clot and the serum separated and collected to be used to assay.

In embodiments in which the sample is a serum or serum derived sample, the sample is generally a fluid sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. The serum is then collected and stored until assayed. Once the patient derived sample is obtained, the sample is assayed to determine the level of GP2 analyte.

The subject sample may be treated in a variety of ways so as to enhance detection of a GP2 analyte. For example, where the sample is blood, the red blood cells may be removed from the sample (e.g., by centrifugation) prior to assaying. Such a treatment may serve to reduce the non-specific background levels of detecting the level of a pancreatic glycoprotein analyte using an affinity reagent. Detection of a GP2 analyte may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30 ™), affinity purification). In some embodiments, the pH of the test and control samples will be adjusted to, and maintained at, a pH which approximates neutrality (i.e. pH 6.5-8.0). Such a pH adjustment will prevent GP2 analyte complex formation, thereby providing a more accurate quantitation of the level of GP2 analyte in the sample. In embodiments where the sample is urine, the pH of the sample is adjusted and the sample is concentrated in order to enhance for detection of the level of a GP2 analyte.

The sample may be assayed to determine the presence and/or amount (i.e., level) of a GP2 analyte using any convenient methodology. In some embodiments, the presence or amount of GP2 analyte is determined by using a GP2 analyte affinity reagent.

As reviewed above, the affinity reagent (i.e. GP2 analyte binding reagent) is a molecule that has a high binding affinity for a GP2 analyte. By high binding affinity is meant a binding affinity of at least about 10⁻⁴ M, usually at least about 10⁻⁶ M or higher, e.g., 10⁻⁹M or higher. The affinity reagent may be any of a variety of different types of molecules, so long as it exhibits the requisite binding affinity for a GP2 analyte.

As such, the affinity reagent may be a small molecule or large molecule ligand. By small molecule ligand is meant a ligand ranging in size from about 50 to about 10,000 daltons, usually from about 50 to about 5,000 daltons and more usually from about 100 to about 1000 daltons. By large molecule ligand is meant a ligand ranging in size from about 10,000 daltons or greater in molecular weight.

The small molecule may be any molecule, as well as binding portion or fragment thereof, that is capable of binding with the requisite affinity to the target protein. Generally, the small molecule is a small organic molecule that is capable of binding to the target analyte of interest. The small molecule will include one or more functional groups necessary for structural interaction with a GP2 analyte, e.g., groups necessary for hydrophobic, hydrophilic, electrostatic or even covalent interactions. Suitable molecules will include functional groups necessary for structural interaction with a GP2 analyte, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups.

Small molecule affinity ligands often comprise cyclical carbon or heterocyclic structures and/or aromatic or polyaromatic structures substituted with one or more of the above functional groups. Also of interest as small molecules are structures found among biomolecules, including peptides, saccharides, fatty acids, steroids, purines, pyrimidines, derivatives, structural analogs or combinations thereof. Such compounds may be screened to identify those of interest, where a variety of different screening protocols are known in the art.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e. a compound diversity combinatorial library. When obtained from such libraries, the small molecule employed will have demonstrated some desirable affinity for a GP2 analyte in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

As pointed out, the affinity reagent can also be a large molecule. Of particular interest as large molecule affinity reagents are antibodies, as well as binding fragments and mimetics thereof. As such, the affinity reagent may be either a monoclonal and polyclonal antibody. In yet other embodiments, the affinity reagent is an antibody binding fragment or mimetic, where these fragments and mimetics have the requisite binding affinity for a GP2 analyte. For example, antibody fragments, such as Fv, F(ab)₂ and Fab may be prepared by cleavage of the intact protein, e.g. by protease or chemical cleavage. Also of interest are recombinantly produced antibody fragments, such as single chain antibodies or scFvs, where such recombinantly produced antibody fragments retain the binding characteristics of the above antibodies. Such recombinantly produced antibody fragments generally include at least the VH and VL domains of the subject antibodies, so as to retain the binding characteristics of the subject antibodies. These recombinantly produced antibody fragments or mimetics of the subject invention may be readily prepared using any convenient methodology, such as the methodology disclosed in U.S. Pat. Nos. 5,851,829 and 5,965,371; the disclosures of which are herein incorporated by reference.

The above described antibodies, fragments and mimetics thereof may be obtained from commercial sources and/or prepared using any convenient technology, where methods of producing polyclonal antibodies, monoclonal antibodies, fragments and mimetics thereof, including recombinant derivatives thereof, are known to those of the skill in the art.

Also suitable for use as an affinity reagent are polynucleic acid aptimers. Polynucleic acid aptamers may be RNA oligonucleotides which may act to selectively bind proteins, much in the same manner as a receptor or antibody (Conrad et al., Methods Enzymol. (1996), 267 (Combinatorial Chemistry), 336-367).

Any convenient assay protocol may be employed. Suitable assays that may be employed include antibody-based assays, e.g. an ELISAs, such as those described in the experimental section infra. Antibody based assays require the use of antibodies, or fragments and mimetics thereof, specific for a GP2 analyte. Of interest are direct assays, i.e., those which employ antibodies, or fragments and mimetics thereof, specific for a GP2 analyte.

Antibodies that specifically bind to a subject GP2 analyte can be prepared using a variety of convenient methods known to those of skill in the art. See Guide to Protein Purification, supra, as well as Antibodies, A Laboratory Manual (Harlow & Lane eds. Cold Spring Harbor Press, 1988). The antibodies may be polyclonal or monoclonal antibodies depending on the nature of the intended use, as long as they are specific for a GP2 analyte.

For preparation of polyclonal antibodies, the first step is immunization of the host animal with a GP2 analyte or an immunogenic fragment, including fragment derivative thereof, where the GP2 analyte immunogen will preferably be in substantially pure form, comprising less than about 1% contaminant. The immunogen may comprise a complete GP2 analyte, fragments or derivatives thereof. To increase the immune response of the host animal, the immunogen may be combined with an adjuvant, where suitable adjuvants include alum, dextran, sulfate, large polymeric anions, oil and water emulsions, e.g. Freund's adjuvant, Freund's complete adjuvant, and the like. The immunogen may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The immunogen is administered to the host, usually intradermally, with an initial dosage followed by one or more, usually at least two, additional booster dosages. Following immunization, the blood from the host is collected, followed by separation of the serum from the blood cells. The Ig present in the resultant antiserum may be further fractionated using known methods, such as ammonium salt fractionation, DEAE chromatography, and the like.

As with the preparation of polyclonal antibodies, the first step in preparing monoclonal antibodies specific for a GP2 analyte and fragments thereof is to immunize a suitable host, where suitable hosts include rats, hamsters, mice and the like, and are preferably mice. The GP2 analyte immunogen, which as above, may be an entire GP2 analyte or a fragment or derivative thereof, is administered to the host in any convenient manner, where such methods include: subcutaneous injection with adjuvants, nitrocellulose implants comprising the immunogen, intrasplenic injections, and the like, where the immunization protocol may be modulated to obtain a desired type of antibody, e.g. IgG or IgM, where such methods are known in the art. Following immunization, plasma cells are harvested from the immunized host, where sources of plasma cells include the spleen, lymph nodes and the like, with the spleen being preferred. The plasma cells are then immortalized with myeloma cells to produce hybridoma cells. A variety of myeloma cell lines are available and known to those of skill in the art. The plasma and myeloma cells are fused by combining the cells in a fusion medium usually in a ratio of about 10 plasma cells to 1 myeloma cell, where suitable fusion mediums include a fusion agent, e.g. PEG 1000, and the like. Following fusion, the fused cells are selected, e.g. by growing on HAT medium. Following hybridoma cell production, culture supernatant from individual hybridomas is screened for reactivity with a GP2 analyte using standard techniques, where such screening techniques include ELISA, dot blot immunoassays and the like. The antibody may be purified from the supernatants or ascites fluid by conventional techniques, e.g. affinity chromatography with a GP2 analyte bound to an insoluble support, protein A sepharose and the like.

The above prepared or obtained antibodies may be modified in a number of different ways to optimize their utility for use in a particular immunoassay. For example, antibody fragments, such as Fv, F(ab)₂ and Fab may be prepared by cleavage of the intact protein, e.g. by protease or chemical cleavage.

The antibodies, fragments or derivatives thereof may also be labeled in order to facilitate detection. A variety of protein labeling schemes are known in the art and may be employed, the particular scheme and label chosen being the one most convenient for the intended use of the antibody, e.g. immunoassay. Examples of labels include labels that permit both the direct and indirect measurement of the presence of the antibody. Examples of labels that permit direct measurement of the antibody include radiolabels, such as ³H or ¹²⁵I, fluorescers, dyes, beads, chemilumninescers, colloidal particles, and the like. Examples of labels which permit indirect measurement of the presence of the antibody include enzymes where a substrate may provide for a colored or fluorescent product. For example, the antibodies may be labeled with a covalently bound enzyme capable of providing a detectable product signal after addition of suitable substrate. Instead of covalently binding the enzyme to the antibody, the antibody may be modified to comprise a first member of specific binding pair which specifically binds with a second member of the specific binding pair that is conjugated to the enzyme, e.g. the antibody may be covalently bound to biotin and the enzyme conjugate to streptavidin. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

The assay of the subject invention may be performed in solution or may use a solid (insoluble) support (e.g. polystyrene, nitrocellulose, or beads), using any standard methods (e.g., as described in Current Protocols in Immunology, Coligan et al., ed.; John Wiley & Sons, New York, 1992). Typical methods include ELISAs (enzyme-linked immunosorbent assays), IRMAs (immunoradiometric assays), and RIAs (radioimmunoassays). Where the assay is performed in solution, the test and control samples are each incubated with a GP2 analyte affinity reagent for a time period sufficient to allow formation of analyte and affinity reagent complexes, preferably between about 0.1 hrs up to 24 hrs, or more. As previously noted, the affinity reagent may include a detectable label (e.g. radionuclide, fluorescer, or enzyme). The sample is then treated to separate the analyte and affinity reagent complexes from excess, unreacted affinity reagent (e.g. by addition of anti-affinity reagent (e.g., anti-immunoglobulin antiserum) followed by centrifugation (e.g., 1000×g for 7 min) to precipitate the analyte and affinity reagent complexes, or by binding to an affinity surface such as a second, unlabelled GP2 analyte affinity reagent (e.g., antibody) fixed to a solid substrate such as Sepharose or a plastic well). Detection of affinity reagent bound to a GP2 analyte may be achieved in a variety of ways well known in the art. If necessary, a substrate for the detectable label may be added to the sample.

Where the assay uses a solid support, the support will have an affinity reagent capable of specifically binding a GP2 analyte, where the affinity reagent is bound to the support surface. The affinity reagent facilitates the stable, wash-resistant binding of a GP2 analyte present in the sample to the solid support. The insoluble supports may be any compositions to which affinity reagents, such as antibodies or fragments and mimetics thereof can be bound, which is readily separated from soluble material, and which is otherwise compatible with the overall method of measuring a GP2 analyte in the sample. The surface of such supports may be solid or porous and of any convenient shape. Examples of suitable insoluble supports to which the affinity reagent is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples. Suitable affinity reagents include antibodies, or fragments and mimetics thereof, which specifically bind a GP2 analyte, or anti-idiotype antibodies, or fragments and mimetics thereof, which specifically bind to the anti-GP2 analyte-antibody. Alternatively, the solid support itself may bind a GP2 analyte directly through the charged properties of the support surface, thus taking advantage of the highly negatively charged nature of a GP2 analyte molecule. Methods for binding affinity reagents (e.g., antibodies, or fragments and mimetics thereof to solid supports are well known in the art. After binding of the affinity reagent to the support, the support may be treated with a blocking agent, which binds to the support in areas not occupied by the affinity reagent. Suitable blocking agents include non-interfering proteins such as bovine serum albumin, casein, gelatin, and the like. Alternatively, several detergents at non-interfering concentrations, such as Tween, NP40, TX100, and the like may be used. Such blocking treatment reduces nonspecific binding.

In certain embodiments, a series of standards, containing known concentrations of GP2 may be assayed in parallel with the samples or aliquots thereof to serve as controls. Generally from about 0.001 to 1 ml of sample, diluted or otherwise, is sufficient, usually about 0.01 ml sufficing. Furthermore, in certain embodiments, each sample and standard will be added to multiple wells so that mean values can be obtained for each. The test and control samples are each incubated with the solid support for a time sufficient for binding of a GP2 analyte to the affinity reagent. The incubation time should be sufficient for a GP2 analyte to bind the insoluble first affinity reagent. Generally, from about 0.1 to 3 hr is sufficient, usually 1 hr sufficing. After incubation, the reacted samples may be washed to remove unbound or non-specifically bound material. Generally, a dilute non-ionic detergent medium at an appropriate pH, generally 7-8, is used as a wash medium. An isotonic buffer, such as phosphate-buffered saline, may be employed in the washing step. From one to six washes may be employed, with sufficient volume to thoroughly wash non-specifically bound proteins present in the sample. Preferably, the washing step will not cause dissociation of GP2 analyte and affinity reagent complexes. A second affinity reagent which specifically binds a GP2 analyte is then incubated with the GP2 analyte-affinity reagent complexes. In some embodiments the second affinity reagent is an anti-GP2 analyte antibody, or fragment and mimetic thereof, where the second affinity reagent preferably binds to an epitope different from the epitope bound by the first affinity reagent. The second affinity reagent (e.g., antibody) used to detect a GP2 analyte bound to the support may be detectably labeled to facilitate direct, or indirect detection of GP2 analyte-first affinity reagent-second affinity reagent complexes. Examples of labels which permit direct measurement of immunocomplexes include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product. In some embodiment, the second affinity reagent (e.g., antibody or fragment and mimetic thereof) is labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such affinity reagent-enzyme conjugates are readily produced by techniques known to those skilled in the art. Alternatively, a third detectably labeled affinity reagent (e.g., antibody, or fragment and mimetic thereof) which specifically binds the second affinity reagent may be used to detect the GP2 analyte-first affinity reagent-second affinity reagent complexes. Examples of third affinity reagent second affinity reagent-specific molecule pairs include antibody/anti-antibody and avidin (or streptavidin)/biotin. Since the resultant signal is thus amplified, this technique may be advantageous where only a small amount of a GP2 analyte is present in the sample. An example is the use of a labeled antibody specific to the second antibody. The volume, composition and concentration of the third affinity reagent solution provides for measurable binding to the GP2 analyte already bound to the second affinity reagent. Generally, the same volume as that of the sample is used: from about 0.001 to 1 ml is sufficient, usually about 0.1 ml sufficing. The concentration will generally be sufficient to saturate the GP2 analyte potentially bound to second reagent.

In such assays, the concentration of the second affinity reagent will generally be about 0.1 to 50 μg/ml, preferably about 1 μg/ml. The solution containing the second antibody is generally buffered in the range of about pH 6.5-9.5. The incubation time should be sufficient for the second affinity reagent to bind available molecules. Generally, from about 0.1 to 3 hr is sufficient, usually 1 hr sufficing. After the second affinity reagent has bound, the insoluble support is generally again washed free of non-specifically bound second receptor, essentially as described for prior washes. After non-specifically bound material has been cleared, the signal produced by the bound conjugate is detected by conventional means. Where an enzyme conjugate is used, an appropriate enzyme substrate is provided so a detectable product is formed. More specifically, where a peroxidase is the selected enzyme conjugate, a preferred substrate combination is H₂O₂ and O-phenylenediamine which yields a colored product under appropriate reaction conditions. Appropriate substrates for other enzyme conjugates such as those disclosed above are known to those skilled in the art. Suitable reaction conditions as well as means for detecting the various useful conjugates or their products are also known to those skilled in the art. For the product of the substrate O-phenylenediamine for example, light absorbance at 490-495 nm is conveniently measured with a spectrophotometer.

Alternatively, a GP2 analyte may be detected by using a competitive binding assay. The test and control samples are incubated with the anti-GP2 affinity reagent as described above, to allow for formation of GP2 analyte-affinity reagent complexes. The affinity reagent may be fixed to a solid surface or in solution. After washing to remove unbound material from the precipitated GP2 analyte-affinity reagent complexes or from the solid support (if any) to which the antibody is fixed, the samples are then incubated with a standard amount of detectably labeled GP2, detectably labeled recombinant hybrid GP2, or a detectably labeled fragment of GP2 which retains the ability to compete with a native GP2 analyte for binding sites on the anti-GP2 analyte binding reagent. Binding is detected by standard means: e.g., by measuring the amount of label associated with (a) the solid support (if any), or (b) the precipitated analyte/binding agent complexes. A lower level of binding of the detectably labeled GP2 in the test sample than in the negative control indicates the presence of an elevated level of GP2 analyte in the test sample.

Alternatively, the binding of the second GP2 molecule used in the competitive binding assay (i.e. the GP2 introduced into the test sample after incubation of the test sample with the anti-GP2 analyte affinity reagent), may be measured by means of an epitope present on the second GP2 molecule which is absent in a GP2 analyte derived from a sample of bodily fluid. For example, the second GP2 molecule may be a recombinant fusion protein which retains the ability to bind competitively to the affinity reagent used in the assay. Binding of GP2 fusion protein to the anti-GP2 affinity reagent may then be detected by incubating the sample with a detectably labeled second affinity reagent which specifically binds the fusion protein and does not bind the GP2 analyte from the sample. An example of a recombinant GP2 fusion protein is one that containing an N-terminal extension of amino acids, which recombinant GP2 fusion protein may be used in such a detection method, since affinity reagents which specifically bind to the N-terminal amino acid extension of the recombinant molecule would not be expected to bind to a GP2 analyte present in a sample. Examples of other epitopes which may be introduced into a GP2 fusion protein include epitopes for use as targets for chemical modification and epitopes which have an altered amino acid sequence relative to a naturally-occurring GP2 analyte (to provide a peptide epitope absent in a GP2 analyte).

Depending on the particular nature of the assay used, it may be desirable to employ affinity reagents that are capable of distinguishing between the various GP2 analyte isoforms. For example, in some embodiments a single type of affinity reagent that recognizes all isoforms of GP2 analytes (e.g., short and long isoforms) may be employed. However, in other embodiments it may be desirable to use different affinity reagents that recognize specific isoforms of GP2 analytes. In such embodiments, the affinity reagents used in the assay will specifically bind to the long isoform of GP2 but will not specifically bind to the short isoform of GP2. Alternatively, in certain embodiments, the affinity reagent used in the assay will specifically bind to the short isoform of GP2 but will not specifically bind to the long isoform of GP2. As such, in some embodiments, the subject assay of the present invention will detect the level of both the short and long isoforms of GP2 in a sample. In other embodiments, the subject assay of the present invention will detect the level of only the long isoform of GP2 in a sample. In yet other embodiments, the subject assay of the present invention will detect the level of only the short isoform of GP2 in a sample.

As summarized above, the subject methods are used to determine at least the presence and often the amount (i.e., level) of a GP2 analyte present in sample from a human subject to determine whether the human subject at least has a pancreatic disease. Specifically, in some embodiments, determining the level of a GP2 analyte in a sample according to the subject methods typically involves comparing the detected signal obtained from the subject methods to a table or other source of predetermined values or reference values (collectively referred to herein as a reference) which provide information about the disease activity in the host, e.g. that positively or negatively correlate to the presence of the pancreatic disease involving abnormal levels of a GP2 analyte, a particular stage of the disease involving abnormal levels of a GP2 analyte, and the like. For example, a table of values may be consulted in this step, where the table comprises representative values for a GP2 analyte as found in patients having at least pancreatic disease (e.g., acute or chronic pancreatitis, or pancreatic cancer) involving abnormal levels of a GP2 analyte. The values may be presented in numerical form, in picture form (e.g. as bands on a gel), and the like. By comparing the observed values with these reference values, e.g. by comparing a pattern of a GP2 analyte in the sample to a reference pattern or picture, characterization of the disease activity, e.g. confirmation of diagnosis, determination of disease state, etc., is readily made.

In other embodiments, determining the level of a GP2 analyte in a sample according to the subject methods involves comparing the level of anti-GP2 affinity reagent binding in the test sample to the level of anti-GP2 affinity reagent binding in the negative and/or positive control samples. In such embodiments the level of affinity reagent binding in the test sample is compared to a range of negative and positive control sample, in which the positive control samples have a range of predetermined quantities of GP2 present, and the negative control samples do not have any GP2 present.

Practice of the subject methods, as described above, results in at least a determination or confirmation that a subject has a pancreatic disease condition. With respect to pancreatitis, e.g., both acute and chronic, the subject methods are more accurate than amylase methods for determining pancreatitis. With respect to the particular amylase assay described in the experimental section, below, the subject methods are at least 1.25 fold more sensitive, such as at least about 1.5 fold or more sensitive, including at least about 2, 5, 7, or 10 fold or more sensitive than the amylase assay, as determined using the sensitivity determination protocols described in the experimental section below. Furthermore, the subject methods have a higher likelihood ratio as compared to amylase assays for pancreatitis diseases, where the likelihood ratio (as determined using the protocols described in the experimental section, below) may be at least about 6, such as at least about 5.5, to about 1.5, including at least about 5, at least about 4, at least about 3.5, at least about 3.0, at least about 2.5, at least about 2, at least about 1.8.

Utility

The subject methods may be used to determine whether a human subject at least has a pancreatic disease. As indicated above, the invention provides methods for assaying a sample from a human subject to determine the presence, and often amount or level of a GP2 analyte present in the sample. The phrase “at least has” is used broadly to refer to any type of information about the state of the pancreatic disease involving abnormal levels or amounts of a GP2 analyte present in a sample from the human subject. As such, the subject methods may be used to facilitate diagnosis of a pancreatic disorder prior to or coincident with the onset of clinical symptoms, confirm an initial diagnosis of a pancreatic disease involving abnormal levels of a GP2 analyte, to determine the state (i.e., severity) of the pancreatic disease in a patient known to have the pancreatic disease involving abnormal levels of a GP2 analyte, to monitor the progression of the pancreatic disease, to predict the occurrence of an attack, and the like.

The method of the subject invention facilitates diagnosis of pancreatic disease prior to or coincident with the onset of clinical symptoms (e.g., abdominal pain, such as epigastric pain). For example, the method of the subject invention may provide a diagnosis of pancreatic disease prior to (e.g., 12-24 hours or even 24-48 hours before) onset of clinical symptoms. Furthermore, the method of the subject invention allows the clinician to provide an accurate diagnosis of pancreatic disease even several hours after cessation of clinical symptoms (e.g., usually up to 5, more usually up to 8, even more usually up to 12, and more usually up to 18 hours), up to one or a few days (e.g., 2 days, usually 3 to 7 days) after cessation of clinical symptoms. The diagnostic method of the subject invention is particularly advantageous over other diagnostic methods for detecting pancreatic disease since, due to its high net negative charge, the half-life of a GP2 analyte in bodily fluids is significantly longer than that of proteins normally used as indicators of a pancreatic disease condition (e.g. detection of amylase, lipase, or trypsinogen for diagnosis of pancreatitis). In one representative embodiment, the subject methods are employed with patients who present with abdominal pain without a diagnosis. Prior to the present invention, a diagnosis of chronic pancreatitis cannot be made in most patients with early disease. Many of these patients present with abdominal pain. The subject methods are useful in those patients with abdominal pain of unclear etiology, where a positive test is employed as a strong indication of a pancreatic source.

In another representative embodiment, the subject methods are employed to assess or determine the severity and extent of pancreatic disease in cystic fibrosis patients. Many patients with cystic fibrosis suffer from chronic pancreatitis that leads to organ failure. An assay is not available to assess pancreatic involvement with cystic fibrosis in its early stages. Accordingly, the subject methods may be employed to determine residual pancreatic function in cystic fibrosis patients.

Where the subject method is employed to confirm an initial diagnosis of a pancreatic disease (e.g., pancreatitis (including acute and chronic) and pancreatic cancer), a sample is obtained from a human subject suspected of having the disease involving abnormal levels of a GP2 analyte. In such embodiments, the subject may be identified as presenting the classical symptoms of pancreatic disease, or have a medical history that indicates susceptibility to pancreatic disease. For example, the sample is assayed for the level of a GP2 analyte present, and then compared to reference values, where the reference values correlate a detected level amount with a pancreatic disease.

Classic symptoms of acute pancreatitis include non-fluctuating midepigastric pain which radiates directly through to the back and may last for many hours to many days. Epigastric pain may be relieved by sitting and leaning forward. Breathing may also be painful if there is an associated pleural effusion and pleuritis. The majority of patients experience nausea and vomiting when the pain reaches its maximum and may experience shock and obtundation if the episode persists for more than several hours. Many patients present with a fever, but have no demonstrable infection. The abdomen is frequently distended and bowel sounds are decreased or absent due to a secondary ileus. Physical examination usually reveals a soft abdomen or only mild voluntary guarding. If severe hemorrhagic pancreatitis has developed, the pain may be excruciating, with marked guarding and even rebound tenderness. In recurrent pancreatitis, a mass may be palpated, indicating the presence of a pseudocyst.

Classic symptoms of chronic pancreatitis include, episodes of severe abdominal pain similar to that in acute pancreatitis develop, although bouts of only moderately severe pain also occur. These episodes of pain typically are separated by asymptomatic periods. In later years, the disease becomes more established, and the episodes of pain may persist or recur daily for weeks or months. Very severe pain may last 2 to 14 days and require continuous administration of narcotics. When greater than 90% of the pancreatic tissue is destroyed, pancreatic insufficiency leads to malabsorption that may be diagnosed by increased fatty stools.

Examples of individuals at a particular risk of developing pancreatic cancer are those whose family medical history indicates above average incidence of pancreatic cancer among family members and/or those who have already developed pancreatic cancer and have been effectively treated who therefore face a risk of relapse and recurrence.

The subject methods are also employed to determine the stage (i.e., severity) of the pancreatic disease involving abnormal levels of a GP2 analyte in the human subject already known to have a pancreatic disease. In other words, the subject method can be used to determine whether the human subject suffering from a pancreatic disease involving an abnormal level of a GP2 analyte is in a remission stage, a chronic stage etc. For example, the subject methods may be employed to determine a clinical remission of pancreatic cancer. To determine the stage of the disease, the observed values (e.g., level) for a GP2 analyte in the assayed sample are compared to reference values that are correlated to a particular stage of disease involving abnormal levels a GP2 analyte.

In some embodiments, the severity of a pancreatic disease may be determined by quantitating the amount of a GP2 analyte in the test sample, or by determining the relative amount compared to standard controls or a reference. For example, quantitation of a GP2 analyte may be achieved by comparing the level of affinity reagent (e.g., antibody, or fragment or mimetic thereof) binding in the test sample to the level of affinity reagent binding in one or more identically treated control samples containing known amounts of a GP2 analyte, or by comparing the test sample signal to a table of standard values (e.g., reference). Where a competitive binding assay is employed, the levels of binding of detectably labeled affinity reagent to a GP2 analyte in a sample from a human subject may be correlated with the levels of binding of the detectably labeled affinity reagent in control samples having a known amount of GP2. The level of a GP2 analyte present in the test sample may then be correlated with a degree of disease severity and patient prognosis by reference to these controls. Quantitation of a GP2 analyte in the test sample may alternatively be achieved by precipitation of the GP2 analyte-affinity reagent complexes from solution and comparison of the level of protein in the test sample precipitate relative to precipitates of control samples having a known amount of GP2.

In yet other embodiments, characterization of disease activity yields information concerning progression of the pancreatic disease in the human subject, e.g. whether progression of the pancreatic disease has accelerated or slowed. For example, the initial characterization date, i.e. the level of a GP2 analyte present in the sample derived from the human subject, could be employed as a baseline value to evaluate subsequent testings, e.g. at some time following the initial testing, e.g. 3 months. If the level of a GP2 analyte decreases in subsequent testing, this indicates that the pancreatic disease is not progressing and may be resolving. Alternatively, if the level of a GP2 analyte increases, this indicates that the pancreatic disease is progressing in severity.

In some embodiments, the subject methods of the present invention may be used in the treatment a human subject for a pancreatic disease. In such embodiments, the subject methods are employed to first determine whether a human subject suffers from a pancreatic disease (or the severity of the disease) by determining the level of a GP2 analyte in a sample derived from the human subject according the subject methods. Once, a determination has been made with respect to whether the human subject suffers from a pancreatic disease, a treatment protocol is identified for the human subject based on the determination of the level of a GP2 analyte in a sample derived from the human subject. Treatment protocols for pancreatic cancer are well know in the art and include, but are not limited to, surgery, chemotherapy, radiotherapy, and the like. Treatment protocols for pancreatitis are well known in the art and include, but are not limited to, surgery, administration of a low-fat diet, administration of fat-soluble vitamins and calcium, administration of insulin to control blood sugar levels, administration of supplemental pancreatic enzyme in order to correct underproduction by the pancreas, and the like.

In other embodiments, characterization data of the level of a GP2 analyte present in a sample derived form a human subject obtained by the subject methods may also be used to determine whether a particular therapeutic regimen is having positive affects with respect to the progression of the pancreatic disease. For example, at various time periods during the course of treatment, the subject methods may be performed to obtain a reading of the amount of a GP2 analyte present in a sample derived form a human subject under a particular treatment regimen. If the level of a GP2 analyte is increasing, this indicates that the treatment regimen is not having the desired effect, where the desired effect is to slow the progression of the pancreatic disease. Alternatively, if the level of a GP2 analyte is decreasing, this indicates that the treatment regimen is working with respect to slowing the progression of the pancreatic disease.

Kits

Also provided are kits that find use in practicing the subject methods, as described above. The kits for practicing the subject methods at least include reagents for assaying a sample derived from a human subject for a GP2 analyte, where such kits may include: GP2 analyte affinity reagents, such as an antibody, or fragment or mimetic thereof, and/or immunoassay devices comprising the same members of a signal producing system, such as antibodies, enzyme substrates, and the like; various buffers for use in carrying out the subject detection assays; a reference for determining the amount of a GP2 analyte in a sample; and the like.

The kits may further include one or more reagents necessary for preparation of the patient derived sample, such as heparin, Ficoll-Hypaque, lysing buffer, protease inhibitor, and the like, e.g. where the patient sample is PBMC derived, etc. In addition, the subject kits may further include one or more components employed in fractionation of the sample, such as an electrophoretic medium or precursors thereof, e.g. dried precursors of polyacrylamide gels, one or more buffer mediums or components thereof, and the like.

In certain embodiments, the kits further include at least an information storage and presentation medium that contains reference data with which assay results may be compared in order to diagnose and/or characterize the pancreatic disease involving abnormal levels of a GP2 analyte in the human subject being assayed, i.e., reference data that that positively or negatively correlate to the presence of the pancreatic disease involving abnormal levels of a GP2 analyte, a particular stage of the disease involving abnormal levels of a GP2 analyte, and the like. The information storage and presentation medium may be in any convenient form, such as a printed information on a package insert, an electronic file present on an electronic storage medium, e.g. a magnetic disk, CD-ROM, and the like. In yet other embodiments, the kits may include alternative means for obtaining reference data, e.g. a website for obtaining the reference data “on-line.”

The kits may further include means for obtaining the patient sample, e.g. a syringe. The subject kits further typically include instructions for carrying out the subject methods, where these instructions may be present on a package insert and/or the packaging of the kit. Finally, the kit may further include one or more reagents from an additional biochemical assay which is used to detect the presence of and/or characterize the pancreatic disease involving abnormal levels a GP2 analyte.

The kit components may be present in separate containers, or one or more of the components may be present in the same container, where the containers may be storage containers and/or containers that are employed during the assay for which the kit is designed.

Systems

Also provided are systems that find use in practicing the subject methods, as described above. The systems for practicing the subject methods at least include reagents for assaying a sample derived from a human subject for a GP2 analyte, where such systems may include: GP2 analyte affinity reagents, such as an antibody, or fragment or mimetic thereof, and/or immunoassay devices comprising the same members of a signal producing system, such as antibodies, enzyme substrates, and the like; various buffers for use in carrying out the subject detection assays; a reference for determining the amount of a GP2 analyte in a sample; and the like.

Furthermore, additional items that are required or desired in the protocol to be practiced with the system components may be present, which additional items include, but are not limited to: means for obtaining the patient sample, e.g. a syringe; one or more reagents necessary for preparation of the patient derived sample, such as heparin, Ficoll-Hypaque, lysing buffer, protease inhibitor, and the like; instructions for carrying out the subject methods; one or more reagents from an additional biochemical assay which is used to detect the presence of and/or characterize the pancreatic disease involving abnormal levels a GP2 analyte.

Devices

Also provided are devices that find use in practicing the subject methods, as described above. Devices for practicing the subject methods at least include reagents for assaying a sample derived from a human subject for a GP2 analyte, where such devices may include: GP2 analyte affinity reagents, such as an antibody, or fragment or mimetic thereof, immobilized on the surface of a solid support.

Additional items that are required or desired in the methods to be practiced with the devices may be present, which additional items include, but are not limited to: means for obtaining the patient sample, e.g. a syringe; one or more reagents necessary for preparation of the patient derived sample, such as heparin, Ficoll-Hypaque, lysing buffer, protease inhibitor, and the like; instructions for carrying out the subject methods using the subject devices; one or more reagents from an additional biochemical assay which is used to detect the presence of and/or characterize the pancreatic disease involving abnormal levels a GP2 analyte.

A number of such devices are known in the art. In one non-limiting example, the apparatus will generally employ a continuous flow-path of a suitable filter or membrane, having at least three regions, a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The measuring region may have bound to it the first affinity reagent, and second labeled affinity reagent combined with the assayed sample and the sandwich assay performed as above.

In another non-limiting example the device is a dipstick, to the surface of which is bound an affinity reagent, such an antibody, or fragment or mimetic thereof, which specifically binds a GP2 analyte. In such an exemplary device, the dipstick is inserted directly into a test sample (e.g., blood, serum, or urine) derived from a human subject for a period of time sufficient to permit binding of a GP2 analyte to the affinity reagent bound to the dipstick. The dipstick may be then withdrawn and, if necessary, washed to remove nonspecifically bound material. The dipstick is then inserted into a container containing a detectably labeled second affinity reagent, such an antibody, or fragment or mimetic thereof, which specifically binds a GP2 analyte. After incubation for a time sufficient for binding of the second antibody to the GP2 analyte-affinity reagent complexes, the dipstick may be washed and binding of the second affinity reagent detected by standard means. Where necessary for detection of the second antibody, the dipstick may be inserted into a second container containing a reagent which activates the detectable label on the second antibody.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following materials and methods are used in the examples below.

Production of anti-human GP2 antibodies

Rabbit polyclonal anti-human GP2 sera was produced as previously described (Fritz et al., Pancreas (2002) 24:336-343). The mouse anti-human GP2 monoclonal antibodies were produced using recombinant human GP2 protein expressed from Chinese Hamster Ovary cells (CHO). The recombinant GP2 protein was produced using the polymerase chain reaction to mutate the human GP2 cDNA such that the amino acids necessary for the formation of the glycosylphosphatidylinositol linkage was deleted and replaced with six histidine residues (Wong et al., 1996, Gene 171:311-312). The resultant cDNA encodes for amino acids #1-505 of human GP2 in addition to the six histidine residues at the carboxyl-terminal end. The cDNA was subcloned into a plasmid expression vector, pDNA3.1, (Invitrogen, Calrsbad, Calif.) that was used to transfect CHO cells using Lipofectamine® (Gibco-Life Technologies, Grand Island, N.Y.). Gentamycin (G418, Gibco-BRL) resistant stable CHO-HGP2 clones were screened for high expression and used to produce soluble secreted human GP2. Human GP2 that was secreted into the media was affinity purified using a nickel-based column according to the manufacture's instructions (Qiagen, Valencia, Calif.). Balb/c mice and New Zealand White rabbits were immunized with the purified His-HGP2 for antibody production. Mouse monoclonal antibodies were produced as previously described (Fritz et al., 2002, Pancreas 24:336-343).

Enzyme Linked Immunosorbent Assay (ELISA)

An ELISA assay was developed using the mouse monoclonal (clone 2G2/E10) and rabbit polyclonal anti-human GP2 antibodies. Polyvinyl microtiter plates (Dynex Technologies Inc., VA) were coated with mouse monoclonal antibody 2G2/E10 and incubated at 4° C. for 16 hr and then blocked with 1% (v/v) bovine serum albumin (Fraction V, Sigma Co., St. Louis, Mo.) in 50 mM Tris-buffered saline (TBS) for 2 hours at 37° C. The plates were washed with phosphate buffered saline (PBS) with 0.05% (v/v) Tween 20 (Sigma Co.) (PBS-T). Each patient sample was serially diluted with buffer (TBS with 0.2% (w/v) BSA, 0.05% (v/v) Tween 20 and 10 mM EDTA) before 100 ml was added to each well. The plates were incubated for 1 hour at 37° C. followed by 3 washes with PBS-T. The plates were then incubated for one hour at 37° C. with the rabbit anti-HGP2 antisera (1:1000 dilution). The plates were washed again with PBS-T before an alkaline phosphatase conjugated goat anti-rabbit IgG antibody (ICN Biochemical, Aurora, Ohio) was added to each well for 1 hour at 37° C. After a final wash, p-nitrophenyl phosphate (Sigma) was used for color development and detected with a microplate reader (Dynatech Laboratories, Chantilly, Va.) at 405 nm. Purified recombinant human GP2 was used as a standard. Calculation of the number of moles of human GP2 was based on a molecular mass of 97 KDa, of which 45% is composed of asparagine-linked glycosylation (Fritz et al., 2002, Pancreas 24:336-343).

Amylase Assay

Amylase was determined in normal and negative control subjects at clinical laboratories at Stanford Hospital or the Veterans Administration Palo Alto Health Care System. Amylase serum levels were assayed in a Beckman Synchron LX analyzer (Beckman Coulter, Inc, Fullerton, Calif.) using maltotetrose as the substrate (Hanson et al., 1979, Clin Chem., 25:1216-1221; Pierre et al., 1976, Clin Chem., 22:1219). The assay is composed of the enzymes maltose phosphorylase, β-phosphoglucomutase, and glucose-6-phosphate dehydrogenase. Amylase that is present in the sample cleaves the substrate, maltotetrose, into maltose. The maltose is phosphorylated by maltose phorphorylase producing β-glucose-1-phosphate. The β-glucose-1-phosphate is then transformed into glucose-6-phosphate by β-phosphoglucomutase. Glucose-6-phosphate and NAD is then transformed by glucose-6-phosphate dehydrogenase to 6-phosphogluconate and NADH. The levels of NADH are measured with absorbance spectrophotometry.

Patient Recruitment and Sample Collection

Patients were recruited from the inpatient wards and outpatient clinics of Stanford Hospital and the Veterans Administration Palo Alto Health Care System (Palo Alto, Calif.). Thirty normal controls consisted of healthy individuals without any history of pancreatic or gastrointestinal disease. A second set of negative controls consisted of 113 patients with no history of pancreatic disease but with other gastrointestinal or non-gastrointestinal problems.

Thirty-one acute pancreatitis patients were recruited for the study. The diagnosis was established using a previously described scoring system based on the presence of abdominal pain, amylase or lipase levels, abdominal computed tomography, and the findings if surgery was performed (Sternby et al., 1996, Mayo Clin. Proc., 71:1138-1144). All acute pancreatitis patients required a score of 3 or greater. Acute pancreatitis patients were staged using clinical and radiologic criteria (Steinberg et al., 1994, N. Eng. J. Med., 330:1198-2110; Balthazar et al., 2002, Radiol. Clin. North Am., 40:1199-1209). Chronic pancreatitis was determined using a scoring system based on the presence of calcifications or pancreatic duct abnormalities as determined by an abdominal CT scan, roentgenogram, or ultrasonagraphy; evidence of pancreatic insufficiency; abdominal pain, weight loss, or glucose intolerance. All 16 chronic pancreatitis patients in this study met the established criteria of 4 or more points (Sternby et al., 1996). Thirty-six patients with pancreatic cancer whose diagnosis was confirmed by pathology were also recruited. Informed consent was obtained from all participants in the study in accordance with the procedures approved by the Stanford University Human Subjects Internal Review Board.

Blood samples were collected into a standard lavender top vacutainer tube containing K₃EDTA. The cellular material was removed with low speed centrifugation. The remaining plasma was recovered and stored at 70° C. until the assay was performed. Serum CA19-9 levels were also recorded if they had been obtained as part of the patient's medical care. Lipase assays were not performed on most patients. Financial constraints did not permit additional laboratory exams such as lipase levels to be obtained for all patients. For 13 hospitalized acute pancreatitis patients, plasma GP2 and amylase levels were obtained daily from the day of admission until the test results were normal or the patient was discharged.

Statistical Analysis

Statistics analysis used StatView 5.0.1 software (SAS Institute, Inc., Cary, N.C.). Statistical comparisons between groups were performed using a non-parametric Mann-Whitney test. Receiver Operator Characteristic (ROC) curves were generated using ROCKIT v. 0.9B (Dr. Charles E. Metz, University of Chicago), which can be obtained at the world wide website of radiology.uchicago.edu/krl/toppage11.htm.

### Example 1

### Development of the GP2 Assay

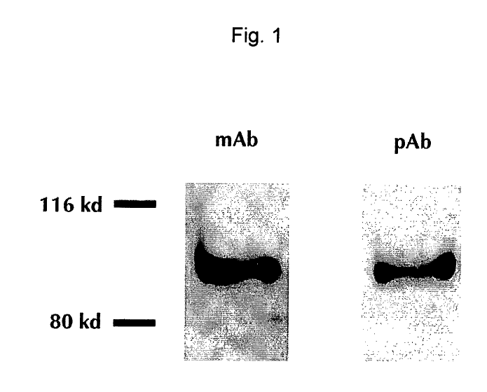

An ELISA assay was developed that utilized two different anti-human GP2 antibodies. The specificity of the antibodies used was determined using protein immunoprecipitation and immunoblotting of human pancreatic secretions, which resulted in a single 97 KDa band consistent with the expected mass of human GP2 (FIG. 1). The developed ELISA assay was linear over a range of at least 0.5-60.6 pmol/L when examined using purified recombinant GP2.

The linearity of the assay was established using purified recombinant human GP2. GP2 levels in normal controls were established using two control populations. Plasma from one group was derived from individuals with no prior significant medical history. A second control group was derived from patients seen at the hospital for reasons other than pancreatic disease. No significant difference in GP2 levels was observed between the two groups.

### Example 2

### Determination of GP2 Plasma Levels in Different Pancreatic Diseases

The assay for human GP2 was tested in a patient population with known pancreatic disease. All three common pancreatic diseases were examined with the GP2 assay.

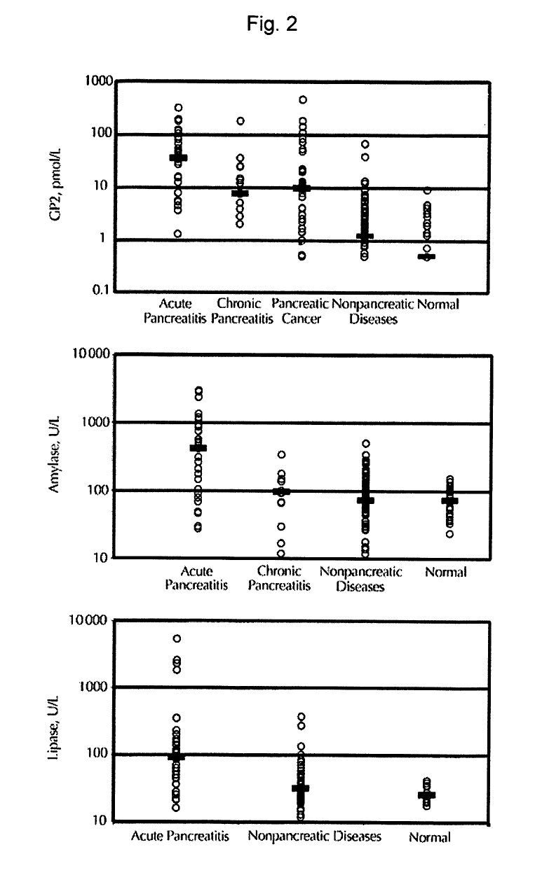

Human subjects recruited into the study included 31 patients with acute pancreatitis, 16 with chronic pancreatitis, 36 with pancreatic cancer, 113 with non-pancreatic diseases, and 30 normal healthy controls (FIG. 2, Table 1). For acute pancreatitis, the etiology was attributed to gallstone disease in 36% of patients and alcohol in 19% of patients (Table 2). No cause was found in 30% of acute pancreatitis patients. For acute pancreatitis, 29 out of the 31 total patients exhibited mild disease by clinical and radiologic criteria. Causes for chronic pancreatitis was determined to be alcohol (75%), gallstone (13%), and cytomegalovirus (6%). No etiology was determined for one patient (6%) with chronic pancreatitis.

The median GP2 level in the 30 normal healthy subjects was not significantly different from the 113 subjects with non-pancreatic disease (p>0.05 by Mann-Whitney test, FIG. 2, Table 3). For patients with acute pancreatitis, chronic pancreatitis, or pancreatic cancer, median GP2 levels were significantly higher than the control groups (p<0.0001, FIG. 2, Table 3).

TABLE 1
Demographic Data

| | | Sex | Mean Age |
|---|---|---|---|
| Groups | Number | (Percent Male) | (Standard Deviation) |
| Normal | 30 | 70.0 | 48.1 |
| | | | (14.5) |
| Non-Pancreatic | 113 | 92.3 | 65.9 |
| Disease | | | (12.8) |
| Acute Pancreatitis | 31 | 51.6 | 46.5 |
| | | | (15.2) |
| Chronic Pancreatitis | 16 | 93.8 | 62.1 |
| | | | (11.6) |
| Pancreatic Cancer | 36 | 72.0 | 64.7 |
| | | | (11.1) |

TABLE 2
Presumed Etiologies for Pancreatitis

| | Number (%) | |
|---|---|---|
| Acute Pancreatitis | | |
| Gallstone/Biliary Sludge | 11 | (36) |
| Alcohol | 6 | (19) |
| Drugs | 1 | (3) |
| Hyperlipidemia | 4 | (13) |
| Unknown | 9 | (30) |
| Chronic Pancreatitis | 16 | |
| Alcohol | 12 | (75) |
| Gallstone | 2 | (13) |
| Cytomegalovirus | 1 | (6) |
| Unknown | 1 | (6) |

TABLE 3
Median GP2 and Amylase Levels in Different Pancreatic Diseases

| | Number | GP2 (pmol/L) | Number | Amylase (IU/L) |
|---|---|---|---|---|
| Normal | 30 | 0.5 | 25 | 74 |
| | | (0.5-4.49) | | (37-121) |
| Non-Pancreatic | 113 | 1.21 | 96 | 74 |
| Disease | | (0.5-6.85) | | (30-199) |
| Acute | 31 | 36.9* | 31 | 420* |
| Pancreatitis | | (5.02-147.88) | | (40-1770) |
| Chronic | 16 | 7.88* | 14 | 98 |
| Pancreatitis | | (2.97-35.27) | | (17-197) |
| Pancreatic | 36 | 9.70* | ND** | ND |
| Cancer | | (0.50-107.91) | | |

Numbers in parenthetical represents the 10^{th}-90^{th} percentile.
*Difference to both control group, normal controls and patients without pancreatic disease was significant (p < 0.001).
**ND: Not determined.

### Example 3

### Amylase Determinations for Different Pancreatic Disease

The median serum amylase level for 25 normal healthy controls was not significantly different from the 96 subjects with non-pancreatic diseases (FIG. 2, Table 3). Amylase levels were determined for all 31 patients with acute pancreatitis, which showed a median significantly higher than the controls (FIG. 2, Table 3). Amylase levels were also available for 14 out of 16 patients with chronic pancreatitis. In contrast to GP2, amylase levels in chronic pancreatitis were not significantly different from normal controls.

### Example 4

### Receiver Operator Characteristic Curves for the GP2 and Amylase Assays

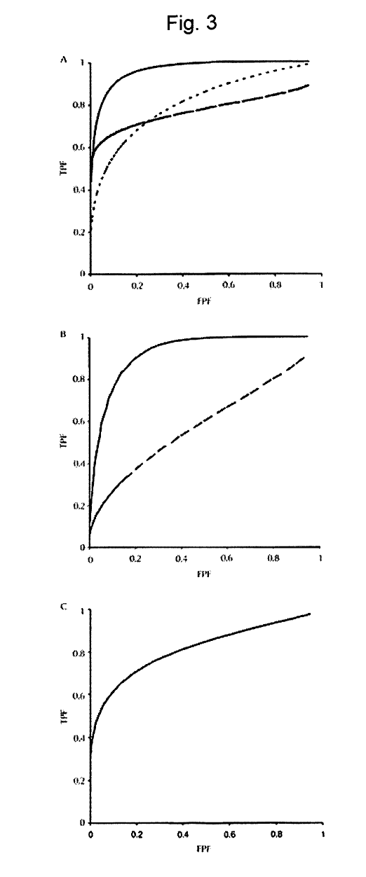

Receiver operator characteristic (ROC) curves were used to assess the overall accuracy of the GP2 and amylase assays. The area under the ROC curve reflects the overall accuracy of a diagnostic test (Hanley et al., 1999, Crit. Rev. Diagn. Imaging 29:307-335). For the GP2 and amylase assays, the area under the ROC curve was greatest for acute pancreatitis followed by chronic pancreatitis (FIG. 3). The area under the curve was significantly greater for the GP2 assay versus the amylase assay for acute pancreatitis (0.96 [95% confidence interval=0.92-0.98] versus 0.77 [0.62-0.88], p<0.005) and chronic pancreatitis (0.92 [0.85-0.96] versus 0.56 [0.37-0.74], p<0.001). The area under the ROC curve for pancreatic cancer was 0.81 [0.70-0.90].

The results demonstrate that GP2 levels exhibited significantly greater accuracy than amylase in determining pancreatic disease in the presence of acute and chronic pancreatitis.

### Example 5

### Sensitivity and Specificity Analysis

Using the ROC curves as a guide, optimal cutoff points were identified for all three pancreatic diseases studied and the sensitivity and specificity were determined. A cutoff of 4.50 pmol/L was established for the GP2 assay. An upper limit of 134 IU/L was used for the amylase assay. The GP2 assay possessed significantly higher sensitivity than amylase (p<0.05 by Chisquare test, Table 4) for acute pancreatitis. Examination of the normal controls revealed no significant differences in specificity between the GP2 and amylase assays for the normal and non-pancreatic disease control groups (Table 4). The specificity of the GP2 assay in the combined negative controls (normal+non-pancreatic disease) was 0.82 (0.76-0.88). The amylase assay specificity was similar to the GP2 assay (Table 4) (p>0.05).

The GP2 assay's sensitivity in patients with chronic pancreatitis was significantly greater than the value determined for the amylase assay (p<0.05, Table 4). The GP2 assay's sensitivity for pancreatic cancer was 0.58 (Table 4). CA19-9 is a commonly used marker for pancreatic cancer (Steinberg et al., 1990, Am. J. Gastroenterol. 85:350-355). Twenty-four pancreatic cancer patients were also tested for CA19-9 and the sensitivity was 0.54 (95% Cl: 0.34-0.74). When GP2 and CA19-9 were combined for 24 patients, the sensitivity improved to 0.72 (0.58-0.87).

TABLE 4
Sensitivity and Specificity of the GP2 and Amylase Assays

| | Sensitivity | | |
|---|---|---|---|
| | (95% Confidence Interval) | | |
| | GP2 | Amylase | P Value* |
| Acute Pancreatitis | 0.94 | 0.71 | <0.05 |
| | (0.85-1) | (0.55-0.87) | |
| Chronic Pancreatitis | 0.81 | 0.43 | <0.05 |
| | (0.62-1) | (0.17-0.69) | |
| Pancreatic Cancer | 0.58 | ND** | ND** |
| | (0.42-0.85) | | |
| Total Pancreatic | 0.76 | 0.51 | <0.01 |
| Disease | (0.67-0.85) | (0.38-0.64) | |
| Normal | 0.90 | 0.92 | >0.05 |
| | (0.79-1) | (0.81-1) | |
| Non-Pancreatic | 0.80* | 0.74* | >0.05 |
| Disease | (0.72-0.87) | (0.65-0.83) | |
| Combined Control | 0.82 | 0.78 | >0.05 |
| Group | (0.76-0.88) | (0.70-0.85) | |

Cutoff points for GP2 was 4.5 pmol/L and for amylase was 134 IU/L.
*Comparing sensitivity between GP2 and amylase tests by Chi Square.
**ND: Not determined.

Significant elevations in GP2 were observed for acute and chronic pancreatitis. A gold standard for the diagnosis of acute pancreatitis currently does not exist. All of the patients fulfilled the diagnostic criteria for acute pancreatitis as described by DiMagno and colleagues (Sternby et al., Mayo Clin. Proc. (1996) 71:1138-1144). Although amylase levels were used for comparison to GP2 and also as a criterion for establishing the diagnosis of acute pancreatitis, all of the patients studied also showed evidence of acute pancreatitis with alternative objective criteria. All patients examined showed evidence of acute pancreatitis on abdominal computed tomography. The majority of acute pancreatitis cases were considered mild using clinical and radiographic staging criteria.

For acute pancreatitis, the GP2 assay's overall sensitivity and specificity were significantly greater than amylase. Assay sensitivities used only patient samples derived from the first blood sample drawn at the time of presentation. The observed amylase sensitivity of 0.71 is lower than that reported by some studies (Steingerg et al., Ann. Intern. Med. (1985) 102:576-580), but higher than that reported by others (Spechler et al., 1983, Dig. Dis. Sci. 28:865-865). Because GP2 levels remained elevated for a longer period than amylase in a subgroup of patients, differences between GP2 and amylase sensitivities would increase if patient samples drawn on the subsequent days of hospitalization were also used for the determinations.

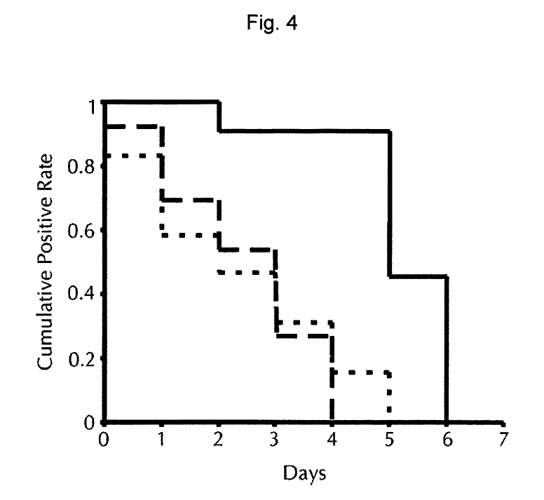

All thirteen patients for which additional blood samples were available on subsequent days of hospitalization expressed GP2 levels that were abnormally elevated at least one day longer than amylase. In addition, peak GP2 levels were often observed at a time when amylase levels were falling (FIG. 4). The results suggest a longer serum half-life for GP2, which would contribute to the increase sensitivity of the GP2 assay.

As revealed by the significantly larger area under the ROC curve, the GP2 assay exhibited greater accuracy than amylase for chronic pancreatitis. All of the patients examined were determined to have ductal abnormalities consistent with chronic pancreatitis as determined by endoscopic retrograde cholangiopancretography, computed tomography, or surgical pathology. Although it is well established that amylase is not a useful marker for chronic pancreatitis, it was compared in this study because of the frequency with which it is usually examined in patients who present to medical attention. The overall sensitivity of GP2 for chronic pancreatitis is better than any other blood assay tested thus far. The GP2 assay is therefore the most sensitive for chronic pancreatitis of mild to moderate severity because the number of acinar cells that represent the source for GP2 will decline with time. It is those patients with early to moderate disease for whom the diagnosis of chronic pancreatitis is often the most difficult to established and an effective blood test that will define this population has yet to be developed.

### Example 6

### Likelihood Ratios

The positive likelihood ratio was calculated for the GP2 and amylase assays. The GP2 assay possessed a higher positive likelihood ratio for acute pancreatitis and chronic pancreatitis than the amylase assay (Table 5). The GP2 assay's likelihood ratio for all pancreatic diseases is 4.7.

TABLE 5
Likelihood Ratios

| | GP2 | Amylase |
|---|---|---|
| Pancreatic Disease | Positive Likelihood Ratio | Positive Likelihood Ratio |
| Acute Pancreatitis | 5.14 | 3.18 |
| Chronic Pancreatitis | 4.46 | 1.92 |
| Pancreatic Cancer | 3.21 | ND* |

Positive likelihood ratio was calculated as sensitivity/(1-specificity).
Specificity used was combined normal and non-pancreatic disease group
*ND: Not determined.

### Example 7

### Time Course of GP2 and Amylase Levels

Serial blood samples were collected on a daily basis for 13 patients with acute pancreatitis from the time of their hospital admission until the day of discharge. Kaplan-Meier survival analysis was used to express the cumulative positive rate for GP2 and amylase (FIG. 4). For 10 of 13 patients, GP2 levels were abnormally elevated longer than amylase. In 3 patients, the amylase and GP2 levels normalized on the same day or both remained abnormally elevated on the day of discharge. Significant differences in the cumulative positive rate were found between the GP2 and amylase assays (p<0.005 by the Mantel-Cox test).

It is evident from the above results that the subject invention provides a exceptionally accurate and easy to perform assay for diagnosing and monitoring or characterizing pancreatic disease conditions in a host. As such, the subject invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of determining whether a human subject at least has a pancreatic disease, said method comprising:
assaying a sample from said human subject for a pancreatic glycoprotein (GP2) analyte to determine whether said human subject at least has said pancreatic disease.

2. The method according to claim 1, wherein said assaying comprises determining the amount of said GP2 analyte in said sample.

3. The method according to claim 2, wherein said amount is determined by comparing a detected signal to a reference.

4. The method according to claim 2, wherein said pancreatic disease is pancreatitis.

5. The method according to claim 4, wherein said method is more accurate than an amylase assay for said pancreatic disease.

6. A method according to claim 4, wherein said pancreatic disease is acute pancreatitis.

7. A method according to claim 4, wherein said pancreatic disease is chronic pancreatitis.

8. A method according to claim 1, wherein said pancreatic disease is pancreatic cancer.

9. A method according to claim 1, wherein said GP2 analyte is human GP2 long isoform.

10. A method according to claim 1, wherein said sample is a blood sample.

11. A method according to claim 1, wherein said assaying employs a GP2 analyte affinity reagent.

12. A method according to claim 11, wherein said affinity reagent is immobilized on a surface of a solid support.

13. A method according to claim 11, wherein said affinity reagent comprises a detectable label.

14. A method according to claim 11, wherein said assaying employs at least two different GP2 analyte affinity reagents.

15. A method according to claim 1, wherein said method is a method of determining severity of said pancreatic disease of said subject.

16. The method according to claim 1, wherein said method is a method of monitoring progression of said pancreatic disease of said subject.

17. A method of treating a human subject for a pancreatic disease, said method comprising:
(a) determining at least whether said human subject suffers from said pancreatic disease by assaying a sample from said human subject for a pancreatic glycoprotein (GP2) analyte; and
(b) identifying a treatment protocol for said human subject based on results from said determining step (a).

18. A kit for use in determining at least whether a subject suffers from a pancreatic disease, said kit comprising:
reagents for assaying a sample for a pancreatic glycoprotein (GP2) analyte; and
a reference.

19. A kit according to claim 18, wherein said GP2 analyte is human GP2 long isoform.

20. A kit according to claim 18, wherein said reagents comprise at least one GP2 analyte affinity reagent.

21. A kit according to claim 20, wherein said at least one GP2 analyte affinity reagent is immobilized on a surface of a solid support.

22. A kit according to claim 18, wherein said kit further comprises a sample obtainment element.

23. A system for use in determining at least whether a subject suffers from a pancreatic disease, said system comprising:
reagents for assaying a sample for a pancreatic glycoprotein (GP2) analyte; and
a reference.

24. A system according to claim 23, wherein said GP2 analyte is human GP2 long isoform.

25. A system according to claim 23, wherein said reagents comprise at least one GP2 analyte affinity reagent.

26. A system according to claim 23, wherein said at least one GP2 affinity reagent is immobilized on a surface of a solid support.

27. A system according to claim 23, wherein said system further comprises a sample obtainment element.

28. A device for use in determining at least whether a subject suffers from a pancreatic disease, said device comprising:
a pancreatic glycoprotein (GP2) analyte affinity reagent immobilized on a surface of a solid support.

29. A device according to claim 28, wherein said GP2 analyte affinity reagent comprises an antibody or binding fragment thereof.
